(19) United States
(12) Patent Application Publication
(10) Pub. No.: US 2020/0054664 A1
(43) Pub. Date: Feb. 20, 2020

(54) METABOLIC SUPPLEMENT FORMULATION AND METHODS OF USE FOR TREATING DEPRESSION AND ANXIETY

(71) Applicant: Germaine Bernard Hawkins, Arlington, TX (US)

(72) Inventor: Germaine Bernard Hawkins, Arlington, TX (US)

(21) Appl. No.: 15/859,098

(22) Filed: Aug. 20, 2018

(51) Int. Cl. A61K 31/714; A61P 25/24; A61K 31/675; A61K 31/519; A61K 31/198; A61K 31/4415; A61K 47/38; A61K 47/12; A61K 9/20; A61P 25/22

(52) U.S. Cl. CPC A61K 31/714; A61P 25/24; A61K 31/675; A61K 31/519; A61P 25/22; A61K 31/4415; A61K 47/38; A61K 47/12; A61K 9/20; A61K 31/198

(57) ABSTRACT

The invention relates to the fields of pharmacology, and provides metabolic supplement formulations and methods for the treatment of depression and anxiety. The metabolic supplement formulation comprises a combination of a vitamin B6 compound, a vitamin B12 compound, a folate, a betaine, N-acetyl-L-tyrosine, and a pharmaceutically acceptable carrier.

### FIELD OF INVENTION

The invention relates to formulations and methods for improving quality of life outcomes based on clinical rating scales.

### BACKGROUND

The World Health Organization (WHO) estimated that mental and neurological diseases affect nearly 700 million people in the world, which represents one third of all cases of non-communicable diseases. Anxiety disorders and depression represent a spectrum of disorders that cause significant psychological distress, interfering with an individual's quality of life, productivity and overall health status. While these disorders can be precipitated or exacerbated by specific environmental stressors, they each also have an explicit biological basis in the nervous system.

It is estimated that only one third of individuals affected by any anxiety disorder receive treatment. This low index is caused by factors such as prejudice, fear, incomprehension, lack of information and inability to seek assistance due to the condition itself. According to the Diagnostic and Statistical Manual of Mental Disorders, Fourth Edition (DSM-IV), anxiety disorders include: panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to conditions, substance-induced anxiety disorder, and anxiety disorder without specific cause.

A collaborative study conducted by the World Health Organization found that anxiety and depression disorders are the most frequent co-occurring psychiatric disorders (Ormel et al. (1994) JAMA 272:1741-1748). According to the study, 45% of patients diagnosed with anxiety disorder also had depression disorder. According to the DSM-IV, depression disorders include major depressive disorder (MDD) or major depression, dysthymic disorder or dysthymia, and minor depression. This category also includes atypical mood disorders, whose development occur under special or singular circumstances, which are psychotic depression, postpartum depression, seasonal affective disorder and bipolar disorder. As used herein, the term “depressive disorder” also includes the anxiety disorders described herein.

Pharmaceuticals that have been developed to address anxiety disorders and depression include the benzodiazepines, which are a broad class of drugs used to treat anxiety, insomnia, seizures, depression, panic attacks, alcohol withdrawal, and other conditions. These drugs act by stimulating GABA benzodiazepine receptors. GABA, which can be considered the brain's “natural valium” is the primary inhibitory neurotransmitter in the central nervous system. By acting as a GABA agonist, benzodiazepines stimulate the receptors to become more sensitive to GABA. In turn, the so-called “GABAergic activity” serves to reduce excitability in the brain, whereas even a mild attenuation of GABA activity can lead to arousal, anxiety, insomnia and restlessness (Nutt & Malizia (2001) Br. J. Psychiatry, 179:390-396). Examples of benzodiazepines include diazepam (Valium) and alprazolam (Xanax).

Other drugs for treating anxiety and/or depressive disorders include tricyclic antidepressants, monoamine oxidase inhibitors, selective serotonin reuptake inhibitors, and serotonin and norepinephrine reuptake inhibitors. While GABA is a major neurotransmitter pathway in the brain, drugs and/or alternative remedies for anxiety and depression can also have beneficial effects by changing the availability of other neurotransmitters in the brain, including serotonin, norepinephrine, and dopamine.

Unfortunately, a significant portion of depressed patients are resistant to treatment with existing antidepressants or combinations thereof either because of non-responsiveness, inadequacy, or because the positive effect wears off to quickly. Troubling side effects may also be seen with existing antidepressants. For example, serotonin-reuptake inhibitors may have significant, unwanted sexual and/or gastrointestinal side effects in both male and female patients. Benzodiazepines also have a number of major limitations: (a) tolerance to their effects rapidly develops with increasing doses becoming required to achieve the same effect; (b) benzodiazepine dependence with chronic use; (c) major withdrawal syndromes, including grand mal seizures, after abrupt discontinuation; (d) overdose; (e) death; (f) high abuse potential; (g) and complications from co-use with alcohol.

Further, 50% or more of patients with depression do not experience an adequate therapeutic response to known administered drugs. In most instances, 2 or more weeks of drug therapy are need before meaningful improvement is observed, as noted in an open-label study on pharmacological treatment of depression. (Rush et al, Am. J. Psychiatry 2006, 163: 1905). There currently is no single effective treatment for depression, anxiety, and other related diseases.

Thus, there remains a need for improved treatments of depression, anxiety and/or other related diseases with compounds that provide increased efficacy and reduced undesirable side effects.

### SUMMARY

A metabolic supplement formulation for the treatment and prevention of anxiety and depression is provided comprising a combination of a vitamin B6 compound, a vitamin B12 compound, Calcium 5-methyltetrahydrofolate, betaine, and N-acetyl-L-tyrosine.

In some embodiments, the combination comprises the vitamin B6 compound in an amount of between 1 mg and 100 mg, vitamin B12 compound in an amount between 100 μg and 2,000 μg, Calcium 5-methyltetrahydrofolate in an amount between 100 μg and 2,000 μg, betaine in an amount between 10 mg and 500 mg, and N-acetyl-L-tyrosine in an amount between 10 mg and 500 mg.

In a particular embodiment, the combination comprises the vitamin B6 compound in an amount of 68 mg, the vitamin B12 compound in an amount of 1,000 μg, Calcium 5-methyl tetrahydrofolate in an amount of 1,000 μg, betaine in an amount of 200 mg, and N-acetyl-L-tyrosine in an amount of 275 mg.

In some embodiments, the vitamin B6 compound is pyridoxal 5′-phosphate.

In other embodiments, the vitamin B12 compound is methyl cobalamin.

In further embodiments, the metabolic supplement formulation further comprises a pharmaceutically acceptable carrier. In particular embodiments, the pharmaceutically acceptable carrier comprises one or more pharmaceutically acceptable excipients. In further particular embodiments, the one or more pharmaceutically acceptable excipients comprise microcrystalline cellulose and/or magnesium stearate.

In another embodiment, a method of treating or preventing anxiety, depression, or another depressive disorder in a subject in need thereof is provided, the method comprising selecting a subject suffering from a depressive disorder, depression, or anxiety, and administering a therapeutically effective amount of any of the metabolic supplement formulations of the invention described herein to the subject.

In other specific embodiments, the invention is a method for lowering depressive feelings, as defined herein, by selecting a subject and administering a therapeutically effective amount of at least one of the formulations of the invention described herein to the subject.

Certain aspects of the presently disclosed subject matter having been stated hereinabove, other aspects will become evident as the description proceeds when taken in connection with the accompanying Examples, Drawings, and References as best described herein below.

### BRIEF DESCRIPTION OF THE DRAWINGS

The invention can be better understood by referring to the following Figures.

FIG. 1 shows initial PHQ-9, initial BAI, 8 weeks PHQ-9, and 8 weeks BAI values collected from the study described in Example 1 collected during the study described in Example 1.

FIG. 2 shows the mean value of initial PHQ-9, initial BAI, 8 weeks PHQ-9, and 8 weeks BAI with standard deviation collected during the study described in Example 1.

FIG. 3 shows the initial PHQ-9 and 8 weeks PHQ-9 value collected during the study described in Example 1.

FIG. 4 shows the initial BAI and 8 weeks BAI value collected during the study described in Example 1.

FIG. 5 shows the maximum, minimum, and quartile value of initial PHQ-9, initial BAI, 8 weeks PHQ-9, and 8 weeks BAI with distribution collected during the study described in Example 1.

FIG. 6 shows the correlation results of initial PHQ-9, initial BAI, 8 weeks PHQ-9 and 8 weeks BAI in the study described in Example 1.

FIG. 7 shows the results of a pair sample t-Test calculated to determine the difference between the variables and result in the study described in Example 1.

FIGS. 8-17 show the results of the study described in Example 2.

### DETAILED DESCRIPTION

The presently disclosed subject matter now will be described more fully hereinafter. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

The presently disclosed subject matter relates to the fields of pharmacology and medicine, and provides metabolic supplement formulations and methods for the treatment of depression and anxiety.

Compositions

In one embodiment, the present invention is directed to a metabolic supplement formulation for the treatment and prevention of anxiety, depression, or another depressive disorder comprising a combination of a vitamin B6 compound, a vitamin B12 compound, a folate, including, but not limited to a folate, a betaine, or its equivalent, and N-acetyl-L-tyrosine. The formulations of the invention are combined and administered within a pharmaceutically acceptable carrier and comprise no other pharmaceutically active agents. In specific embodiments of the invention, the formulations comprise about 545 mg active ingredients.

Any betaine may be used in accordance with the invention, including betaine anhydrous and betaine hydrochloride, or other salts of betaine. In one specific embodiment, the invention comprises betaine anhydrous.

It should be noted that “treating” or “treatment” refer to the act of administering the formulation of the invention to a subject in need thereof in an amount therapeutically effective in reducing feeling of depression, or in in an amount that increases the subjects neurotransmitter levels such that the subjects experience an improved mood state. A reduction of feelings of depression, or depressive feeling, as used herein, may be determined by various methods, including PHQ-9 and BHI self-reported values. The formulations of the invention and methods of using the formulations increase neurotransmitter levels using various mechanisms. Using multiple mechanisms to increase neurotransmitter loads produces a synergistic effect on the speed and efficacy. Subject feel better, sooner than would be expected when taking only one of the elements of the invention.

The formulations of the invention also decrease homocysteine levels. Increased levels of homocysteine have been shown to increase a subject's risks of heart attack and have been associated with high anxiety and depression. Vitamins B9, B12 and B6 act to disrupt the production of homocysteine levels in the human body and also act as precursors to the production of dopamine and other neurotransmitters important to mood stabilization and anxiety control.

While all the components of the formulation of the invention are known to those of skill in the art, no one has combined them sufficient to affect a synergistic effect in the reduction of depressive feelings in subjects suffering from anxiety, depression, or other depressive disorders. The formulations discovered show increased efficacy in the particular formulations and ratios described herein. The results are surprising when the prior art is examined as the effect of the combination is greater than the sum of each, when individual efficacy is studied.

In particular embodiments, the combination comprises the vitamin B6 compound in an amount of between 1 mg and 100 mg, the vitamin B12 compound in an amount between 100 μg and 2,000 μg, the Calcium 5-methyltetrahydrofolate in an amount between 100 μg and 2,000 μg, the betaine in an amount between 10 mg and 500 mg, and the N-acetyl-L-tyrosine in an amount between 10 mg and 500 mg. While all of these substances are well known in the art, no one has discovered a particular formulation or combination of them sufficient to demonstrate a significant and measurable synergistic effect in reducing depression and anxiety symptoms in animals, including humans, until the presently claimed formulations and methods. Two clinical studies were conducted to assess the efficacy of the formulation of the invention. Both show significantly sufficient lowering of depression and anxiety symptoms in patients not currently taking any new medicines to treat depression or anxiety.

Tyrosine has been used to boost dopamine production. Tyrosine, however, is inactive alone and requires activation by an available acetyl group. The claimed methods and formulations include N-acetyl-L-tyrosine. The acetyl group activates tyrosine so that it is available for use within the body sooner than formulations that employ tyrosine. The body is able to metabolize and convert N-acetyl-L-tyrosine into dopamine quicker and more efficiently than tyrosine.

In a specific embodiment, referred to in Examples 1 and 2 as ADVYNDRA, the formulation is a combination that comprises the vitamin B6 compound in an amount of 68 mg, the vitamin B12 compound in an amount of 1,000 μg, the Calcium 5-methyltetrahydrofolate in an amount of 1,000 μg, the betaine in an amount of 200 mg, and the N-acetyl-L-tyrosine in an amount of 275 mg. The formulation is housed in any acceptable pharmaceutical carrier, and has a total of 545 mg of active ingredients. The formulation includes no other pharmaceutically active agents. As used herein, this specific embodiment of the invention is referred to as “ADVYNDRA.”

The phrase, “vitamin B6 compounds” means pyridoxine, pyridoxal, pyridoxomaxine, their phosphorylated derivatives pyridoxine 5′-phosphate, pyridoxal 5′-phosphate, and pyridoxamine 5′-phosphate. In a particular embodiment, the vitamin B6 compound is pyridoxal 5′-phosphate.

The phrase, vitamin B12 compounds” as used herein means a class of compounds which includes vitamin B12 and its analogues, derivatives or conjugates. The class of vitamin B12 compounds includes cyanocobalamin (CN-Cbl), aqua cobalamin, adenosylcobalamin, methyl cobalamin, hydroxycobalamin (HC), cyanocobalamin carbanalide, and 5-o-methylbenzylcobalmin [(5-OmeB-za)CN-Cbl] as well as the desdimethyl, monoethylamide and the methylamide analogues of all of the above. Also included are the various analogues and homologues of cobamamide such as coenzyme B12 and 5-deoxydenosylcobalamin. Other analogues include chlorocobalamin, sulfitocobalamin, nitrocobalamin, thiocyanatocobalamin, benzimidazole derivatives such as 5,6-dichlorobenzimidazole, 5-hydroxybenzimidazole, trimethylbenzimidazole, as well as adenosylcyanocobalamin [(Ade)CN-Cbl], cobalamin lactone, cobalamin lactam and the anilide, ethylamide, monocarboxylic and dicarboxylic acid derivatives of vitamin B12 or its analogues.

Preferred derivatives of vitamin B12 include the mono-, di- and tricarboxylic acid derivatives or the proprionamide derivatives of vitamin B12. In addition, the compositions include polymers of these analogues or vitamin B12 conjugated to other molecules or encapsulated. The singular form, “vitamin B12 compound”, means any one or more compounds from the class of vitamin B12 compounds. In a particular embodiment, the vitamin B12 compound is methyl cobalamin.

The term, “betaine” is also known as betaine anhydrous, trimethylglycine, N-trimethylglycine, glycine betaine, glycol betaine, oxyneurine and lycine. Its chemical name is 1-carboxy-N,N,N-trimethylmethanaminium inner salt. The molecular formula of betaine is C₅H₁₁NO₂, its chemical formula is (CH₃)₃N⁺—CH₂COO⁻. The hydrochloride of betaine is known as betaine hydrochloride, betaine HCl, and pluchine. Its chemical name is 1-carboxy-N,N,N-trimethylmethanaminium chloride.

Betaine-homocysteine methyltransferase (BHMT) is a zinc metalloenzyme that catalyzes the transfer of a methyl group from betaine to homocysteine in the formation of methionine. BHMT is found in the liver and kidneys and may also exist in brain tissue. Betaine can lower homocysteine levels in some persons with primary hyperhomocysteinemia/homocystinuria via this enzyme. Betaine has also been found to lower homocysteine levels in some animal studies, again, via BHMT. Subjects provide with the claimed formulations have been shown to start to feel better quicker, i.e. in about one week, when compared to subjects treated with formulations that are currently available.

Betaine equivalents are defined as betaine, dimethylglycine, betaine inner salts in anhydrous and hydrated forms as well as the forms wherein the molecule is associated with inorganic acids like hydrochloric acid or organic acids like citrate, malate aspartate or sarcosine. In a preferred embodiment betaine, dimethylglycine or a combination thereof is used. In a most preferred embodiment betaine is used. As used herein, and in one embodiment of the invention, the phrase “betaine equivalents” is used to describe betaine and its equivalents. In specific embodiments of the invention, betaine equivalents are employed. In other specific embodiments of the invention, betaine is employed in the invention without a betaine equivalent.

In accordance with the invention, any folate may be employed. However, methyl folates are preferred because an available methyl group is required to activate the folate. This immediate activation seen when methyl folates are employed in the formulations and methods of the invention, results in higher efficacy and quicker on-set of increase mood state. In specific embodiments of the invention, methyl folates are employed. In other more specific embodiments, the formulations and methods comprise Calcium 5-methyl tetrahydrofolate.

Among folates and reduced folates, the calcium salts can be mentioned as the most relatively stable derivatives: U.S. Pat. Nos. 5,817,659 and 6,441,168 disclose crystalline salts, preferably calcium salts, of 5-methyl-(6R, S)-, -(6S)- or -(6R)-tetrahydrofolic acid having a water of crystallization of at least one equivalent per equivalent of said acid. Calcium 5-methyl tetrahydrofolate is the only folic acid derivative on the market which is able directly to penetrate the blood/brain barrier without further metabolism. Naturally occurring 5-methyltetrahydrofolic acid is solely in the S form; the R form is biochemically inactive and is excreted through the kidney. N-acetyl-L-tyrosine is a precursor of norepinipherine, a neurotransmitter. N-acetyl-L-tyrosine has the IUPAC name (2S)-2-acetamido-3-(4-hydroxyphenyl) propionic acid.

In addition to the active ingredients, the presently disclosed compositions may contain suitable pharmaceutically acceptable carriers comprising pharmaceutically acceptable excipients and auxiliaries which facilitate processing of the active agents into preparations which can be used pharmaceutically. The term “pharmaceutically acceptable excipient” as used herein means one or more compatible solid or liquid filler, diluents or encapsulating substances that are suitable for administration into a subject. In particular embodiments, the one or more pharmaceutically acceptable excipients comprise microcrystalline cellulose and/or magnesium stearate.

Use of pharmaceutically acceptable inert carriers to formulate the active agents herein disclosed for the practice of the disclosure into dosages suitable for systemic administration is likewise within the scope of the disclosure. The active agents can be formulated readily using pharmaceutically acceptable carriers well known in the art into dosages suitable for oral administration. Such carriers enable the active agents of the disclosure to be formulated as tablets, pills, capsules, liquids, gels, syrups, slurries, suspensions and the like, for oral ingestion by a subject (e.g., patient) to be treated.

Pharmaceutical preparations for oral use can be produced by any known method, including by combining the active agents with solid excipients, optionally grinding a resulting mixture, and processing the mixture of granules, after adding suitable auxiliaries, if desired, to obtain tablets or dragee cores. Suitable excipients are, in particular, fillers, such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof, such as sodium alginate.

Other acceptable carriers, excipients, or stabilizers are nontoxic to recipients at the dosages and concentrations employed, and can include buffers such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers, such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or nonionic surfactants such as Tween, Pluronics, or PEG. Other suitable carriers include aqueous and oleaginous carriers such as, for example, white petrolatum, myristate, lanolin, mineral oil, fragrant or essential oil, nasturtium extract oil, sorbitan mono-oleate, detergents (e.g., polyoxyl stearate or sodium lauryl sulfate) and mixed with water to form a lotion, gel, cream, or paste, semi-solid composition.

Additional suitable carriers comprise water-in-oil or oil-in-water emulsions and mixtures of emulsifiers and emollients with solvents such as sucrose stearate, sucrose cocoate, sucrose distearate, mineral oil, and water. For example, emulsions containing water, glycerol stearate, glycerin, mineral oil, synthetic spermaceti, butylparaben, propylparaben and methylparaben are commercially available. Preservatives may also be included in the carrier including methylparaben and propylparaben. The composition may also include a plasticizer such as glycerol. The composition of the carrier can be varied so long as it does not interfere significantly with the pharmacological activity of the active ingredients of the therapeutic composition.

The preparation of pharmaceutical formulations is well known in the art and has been described in many articles and textbooks. (See e.g., Gennaro A. R. ed. (1990) Remington's Pharmaceutical Sciences, Mack Publishing Company, Easton, Pa., and especially pages 1521-1712 therein. Known methods will not be restated herein.

Methods of Treatment

Once formulated, metabolic supplement formulations of the presently disclosed subject matter may be used without limitation to treat anxiety and/or depression in subjects in need thereof. The method comprises administering a therapeutically effective amount of any of the metabolic supplement formulations disclosed herein to the subject, which has been selected based on related diagnoses. It should be understood that patient selection may be done by a professional who is not directly administering the formulations of the invention.

Anxiety disorders may include panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to general medical condition, substance-induced anxiety disorder and anxiety disorder without specific cause.

Depression disorders may include major depression disorder, dysthymia disorder, non-specific depression disorder, psychotic depression, postpartum depression, seasonal affective disorder and bipolar disorder.

The phrases “effective amount,” and “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disorder or condition, or one or more symptoms thereof; prevent the advancement of a disorder or condition cause the regression of a disorder or condition; prevent the recurrence, development, onset or progression of a symptom associated with a disorder or condition, or enhance or improve the prophylactic or therapeutic effect(s) of another therapy.

A physician having ordinary skill in the art can readily determine and prescribe the effective amount of the presently disclosed composition required. Accordingly, the dosage range for administration will be adjusted by the physician as necessary. For example, optimal dosing schedules can be calculated from measurements of drug accumulation in the body of the subject and on basis of, e.g., the present examples. Persons of ordinary skill can easily determine optimum dosages, dosing methodologies and repetition rates.

In general, dosage is calculated according to body weight, and may be given once or more daily, weekly or monthly. The dosage of any formulations of the present invention will also vary depending on the symptoms, age and body weight of the patient, the nature and severity of the disorder to be treated or prevented, the route of administration, and the form of the subject composition. Any of the subject formulations may be administered in a single dose or in divided doses. The form of the depression condition being treated, the length of treatment time desired, the age and the condition of the patient, are also considered by the attending physician determining dosage. It should be understood that the specific formulations stated herein may be altered based on these factors, as well.

In various embodiments, the present invention may be administered by various means, depending on intended use, as is well known in the art. For example, if formulations of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations, or suppositories. For application by the ophthalmic mucous membrane route, compositions of the present invention may be formulated as eye drops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

As used herein, the term “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the diagnosis or treatment of an existing disorder or condition or the prophylactic diagnosis or treatment for preventing the onset of a disorder or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disorder or condition. Thus, the terms “subject” and “patient” are used interchangeably herein. Subjects also include animal disease models (e.g., rats or mice used in experiments, and the like).

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1: Safety and Effectiveness of ADVYNDRA Metabolic Supplement in Improving Quality of Life Outcomes Based on Clinical Rating Scales Measuring Depression and Anxiety Symptoms

The change in total PHQ-9 and BAI scores between baseline and endpoint were the primary outcomes measures. PHQ-9 and BAI scores are each indicators of depressive feelings. On both scales, higher scores indicate poorer outcomes, or higher depressive feelings among subjected tested. It should be understood that the phrase, “reduce depressive feelings,” or “reduction in depressive feelings” means that the PHQ-9 and BAI scores are lower or lowering, as reported by patients who are suffering from depressive disorders, or who have suffered from depressive disorders. These measures are based on self-reported, rated inventory of depressive and anxiety symptoms. All items are scored on a scale of zero to 3 for both scales, and the sum of the scores provide total scores for the measures. Scores can range from 0-27 for PHQ-9 and 0-63 for BAI.

The 8-week study was designed to determine whether ADVYNDRA is safe and effective in improving quality of life outcomes. Participants were given either ADVYNDRA or a placebo for 8 weeks. Depression scales and self-report questionnaires were used to assess participants. All participants received 3 months of free ADVYNDRA and clinic visits as necessary.

Inclusion Criteria

A total of 27 adults between ages 18 and 75 that reported feeling so depressed and anxious that their quality of life was impacted where chosen as participants. If on psychotropics, no new antidepressants/mood stabilizers or changes in dosing of current antidepressants/mood stabilizers were allowed to be taken within the previous 4 weeks before entering study.

Exclusion Criteria

Participants who were concurrently on herbal mood enhancing supplements, including but not limited to St. John's Wort, SAM-e, pregnant, nursing or breastfeeding, or who had recent use of marijuana, alcohol or other illicit substances where excluded from the study. Those with prior diagnosis of Bipolar, Schizophrenia or other psychotic disorders; uncontrolled—BP of >140/90 mmHg while on antihypertensives or untreated and above 150/90 were also excluded from participation in the study.

Study Design

Participants were given a bottle of ADVYNDRA, each containing 60 capsules. Patients were instructed to take two capsules daily 20 minutes before first morning meal. At the 4-week visit, patients received the second bottle with 30-day supply of medication.

Screening

Selected 27 adult patients based on above inclusion and exclusion criteria.

Day 1: Baseline urine drug toxicology, PHQ-9 and BAI.

Week 4: PHQ-9 and BAI screen for adverse events, intolerable side effects; drug toxicology; pill count for patient adherence; supply additional 30 days of medication.

Week 8: PHQ 9 and BAI screen for adverse events, intolerable side effects.

Result and Discussion

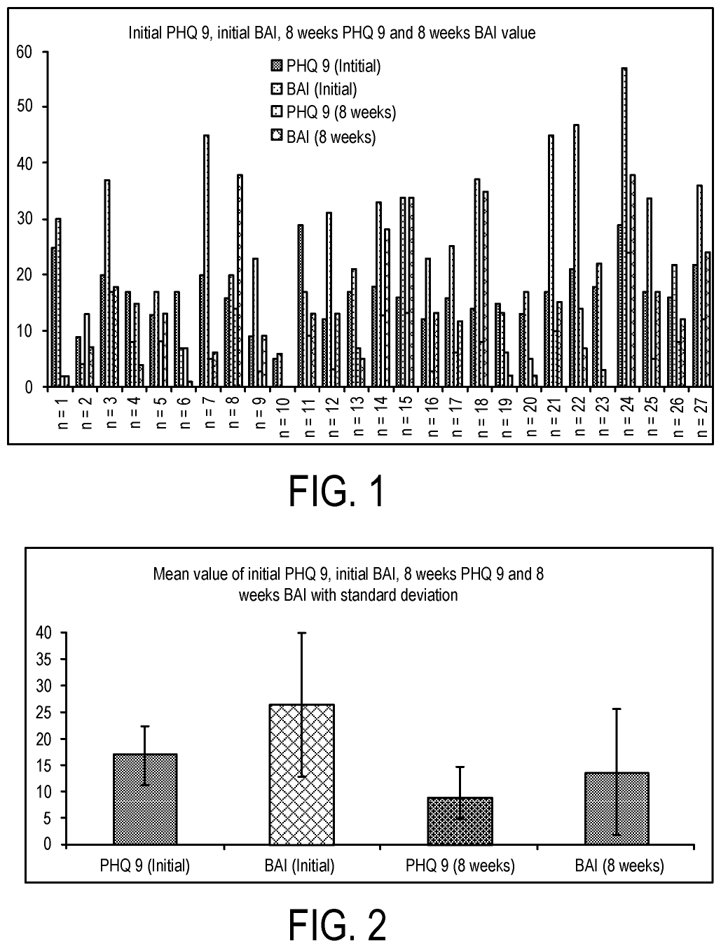

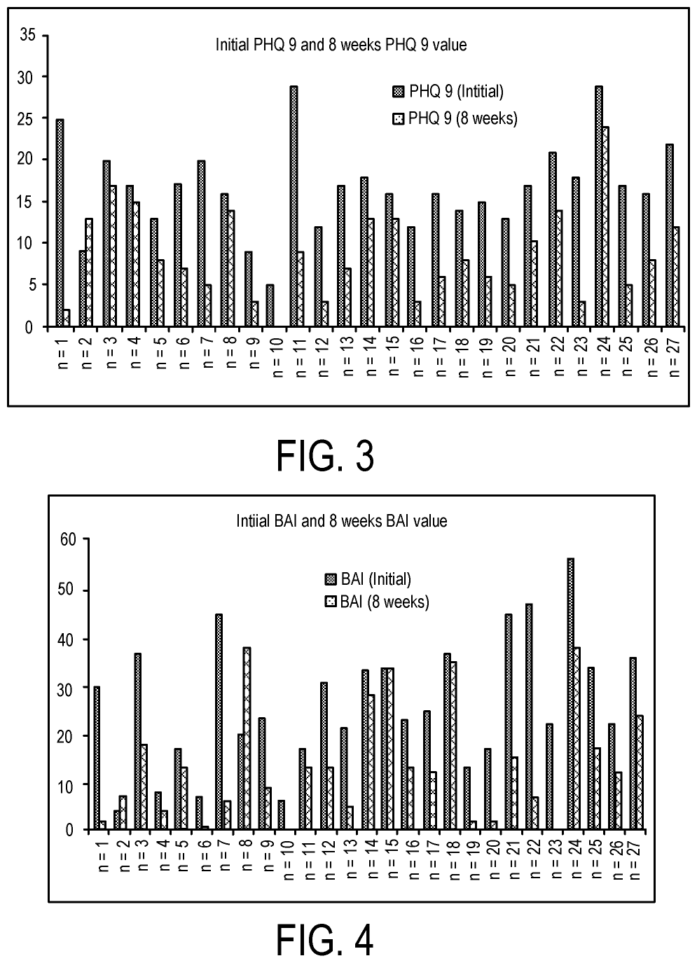

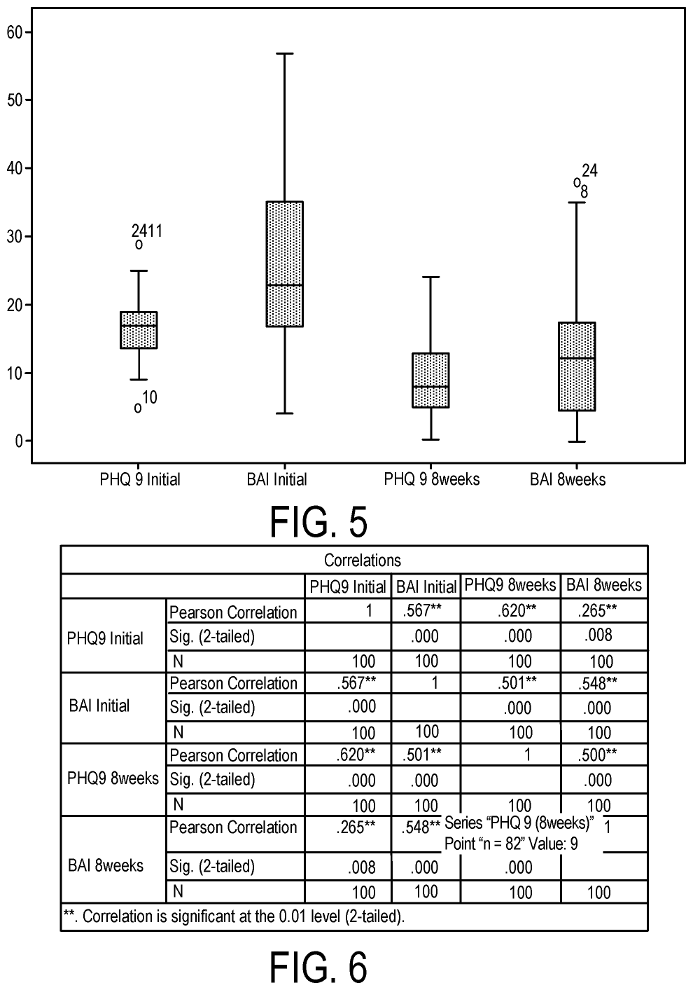

The initial PHQ-9, initial BAI, 8 weeks PHQ-9, and 8 weeks BAI value are shown in FIG. 1. The mean value of initial PHQ-9, initial BAI, 8 weeks PHQ-9, and 8 weeks BAI with standard deviation are shown in FIG. 2. The initial PHQ-9 and 8 weeks PHQ-9 value are shown in FIG. 3. The initial BAI and 8 weeks BAI value are shown in the FIG. 4. The maximum, minimum, and quartile value of initial PHQ-9, initial BAI, 8 weeks PHQ-9, and 8 weeks BAI with distribution are shown in FIG. 5.

The correlation of initial PHQ-9, initial BAI, 8 weeks PHQ-9 and 8 weeks BAI is shown in FIG. 6. The results indicate a strong positive correlation between PHQ-9 Initial with BAI Initial (r=0.529, p=0.005), BAI Initial with BAI 8 weeks (r=0.524, p=0.005) and PHQ-9 8 weeks with BAI 8 weeks (r=0.604, p=0.001). There is a moderate positive correlation between PHQ-9 Initial with PHQ-9 8 weeks (r=0.480, p=0.011) and BAI Initial with PHQ-9 8 weeks (r=0.387, p=0.046). But there is no significant correlation PHQ-9 Initial with BAI 8 weeks (r=0.256, p=0.197).

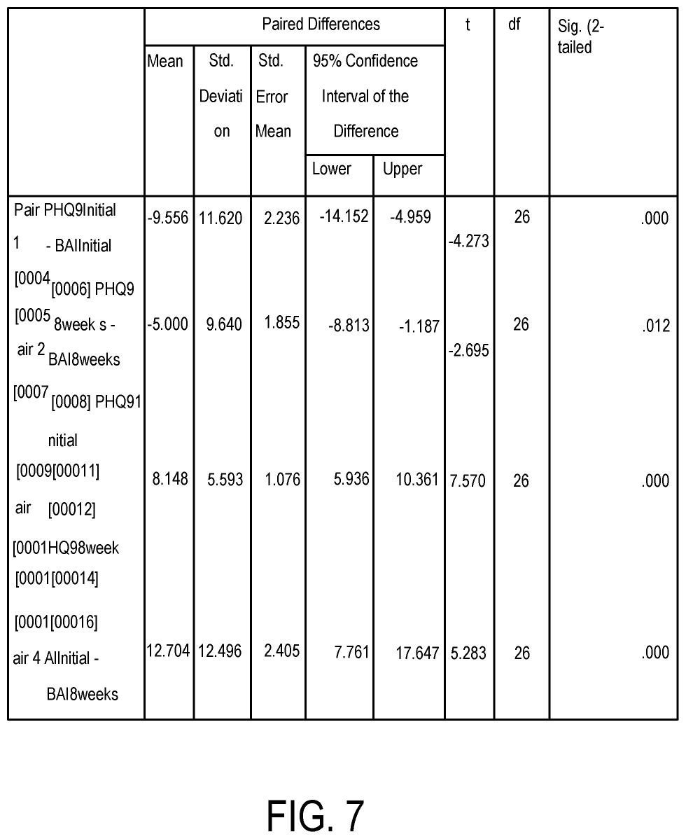

The results of a pair sample t-Test calculated to determine the difference between the variables and result is shown in FIG. 7. The t-stat value of initial PHQ-9 and initial BAI is 4.273 and the significance level is 0.000 (which is <0.05). So, there is a statistically significance difference between initial PHQ-9 and initial BAI. The t-stat value of 8 weeks PHQ-9 and 8 weeks BAI is 2.695 was at a significance level of 0.012 (which is <0.05). So, there is also a statistically significance difference between 8 weeks PHQ-9 and 8 weeks BAI. Moreover, the t-stat value of initial PHQ-9 and 8 weeks PHQ-9 is 7.570 and the significance level is 0.000 (which is <0.05). So, there is a statistically significance difference between initial PHQ-9 and 8 weeks PHQ-9. Again, the t-stat value of initial BAI and 8 weeks BAI is 5.283 and the significance level 0.000 (which is <0.05). So, there is a statistically significant difference also between initial BAI and 8 weeks BAI.

Conclusion

The experimental results show that there is a statistically significant difference between the initial PHQ-9 and 8 weeks PHQ-9, and between initial BAI and 8 weeks BAI. One of skill in the art would therefore conclude that ADVYNDRA, and related formulations are safe and effective in improving quality of life outcomes by reducing symptoms of depression and anxiety in patients.

### Example 2: Safety and Effectiveness of ADVYNDRA Metabolic Supplement in Improving Quality of Life Outcomes Based on Clinical Rating Scales Measuring Depression and Anxiety Symptoms

An 8 week, open label, clinical trial was conducted to assess the efficacy of ADVYNDRA in improving overall quality of life as measured by BAI and PHQ-9. The study was interventional and for the primary purpose of treating human patients. The primary outcome measures were the overall change in total PHQ-9 and BAI scores between a patient's baseline, self-reported inventory, and the patients self-reported inventory after 8 weeks. All measures were scored on a scale of 0-3, and the sum of the scores provided total scores for the analysis from 0-27 for PHQ-9 and 0-63 for BAI. On both scales, higher outcomes indicated poorer outcomes, or greater feelings of depression and/or anxiety.

Inclusion Criteria:

A total of 100 patients, between ages 18 and 65 that reported feeling depressed and anxious impacting their quality of life occupationally, academically and/or socially. If on psychotropics, no new antidepressants/mood stabilizers or changes in dosing of current antidepressants/mood stabilizers within previous 4 weeks of entering study.

Exclusion Criteria:

Patients were excluded that were currently on herbal mood enhancing supplements, including but not limited to St. John's Wort, SAM-e, pregnant, nursing, or breastfeeding, or currently or recently using marijuana, alcohol or other illicit substances. Prior diagnosis of Bipolar. Schizophrenia or other psychotic disorders; uncontrolled—BP of >140/90 mmHg while on antihypertensives or untreated and above 150/90 also excluded patients from the study.

Study Design

All patients were given a bottle of ADVYNDRA, each containing 60 capsules. Patients were instructed to take two capsules daily 20 minutes before first morning meal. At the 4-week visit, patients will receive a second bottle with a 30-day supply of medication.

Screening:

Selected 100 adult patients based on above inclusion and exclusion criteria.

Week 1: Baseline urine drug toxicology, PHQ-9 and BAI

Week 2: PHQ-9 and BAI/screen for adverse events, intolerable side effects

Week 4: PHQ-9 and BAI screen for adverse events, intolerable side effects; drug toxicology; pill count for patient adherence; supply additional 30 days of medication

Week 8: PHQ-9 and BAI

Post Week 8 Assessments

DATA COLLECTION for the study was outsourced.

Result and Discussion

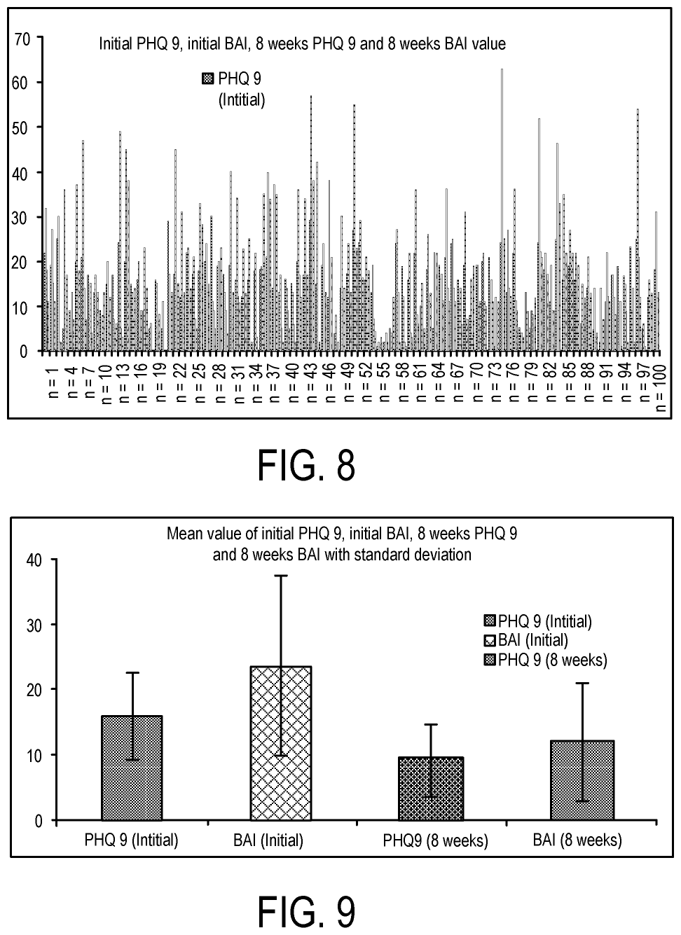

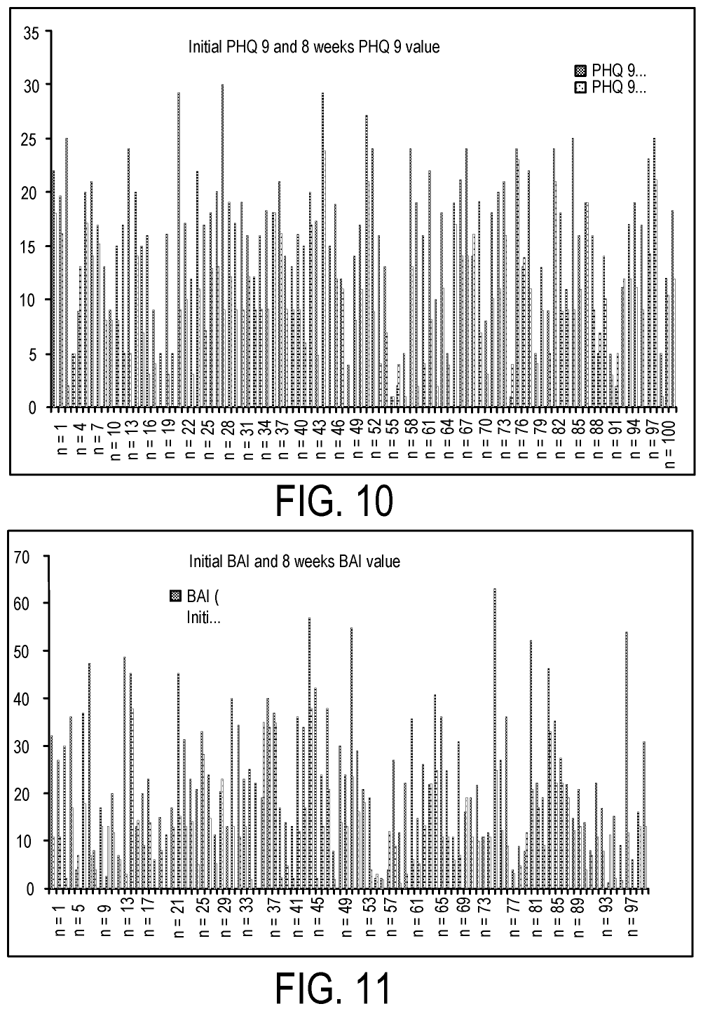

The initial PHQ-9, initial BAI, 8 weeks PHQ-9 and 8 weeks BAI value is shown in the FIG. 8. The mean value of initial PHQ-9, initial BAI, 8 weeks PHQ-9 and 8 weeks BAI with standard deviation is shown in the FIG. 9. The initial PHQ-9 and 8 weeks PHQ-9 value is shown in FIG. 10. The initial BAI and 8 weeks BAI value is shown in FIG. 11.

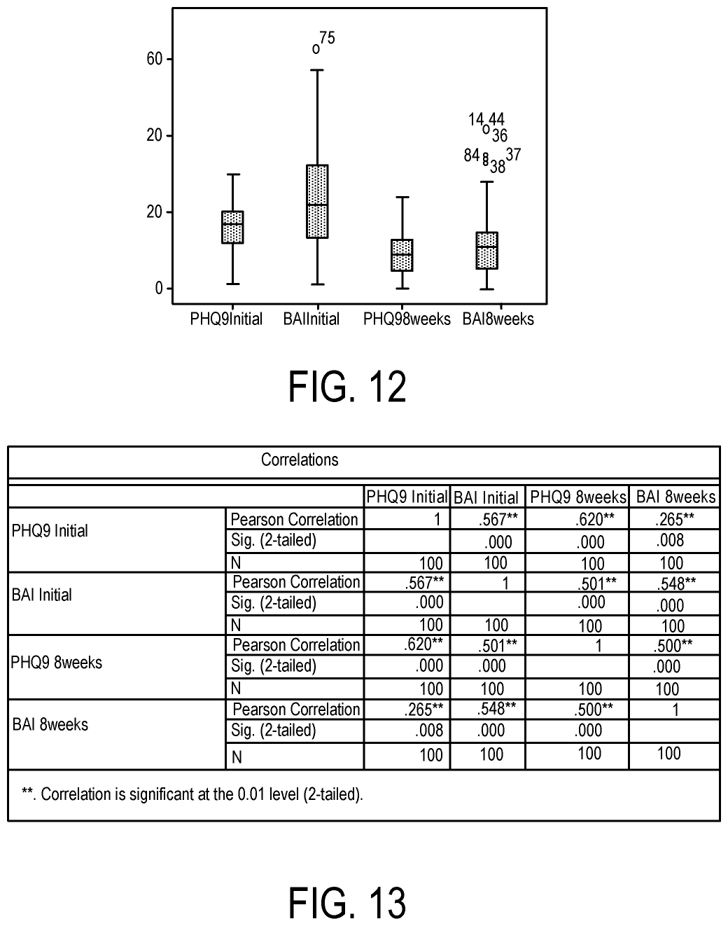

The maximum, minimum and quartile value of initial PHQ-9, initial BAI, 8 weeks PHQ-9 and 8 weeks BAI with distribution in FIG. 12.

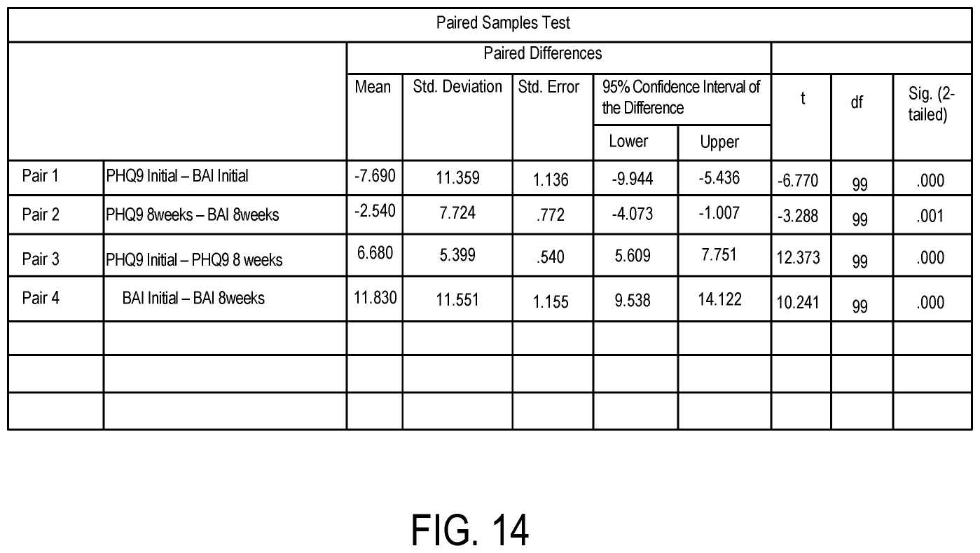

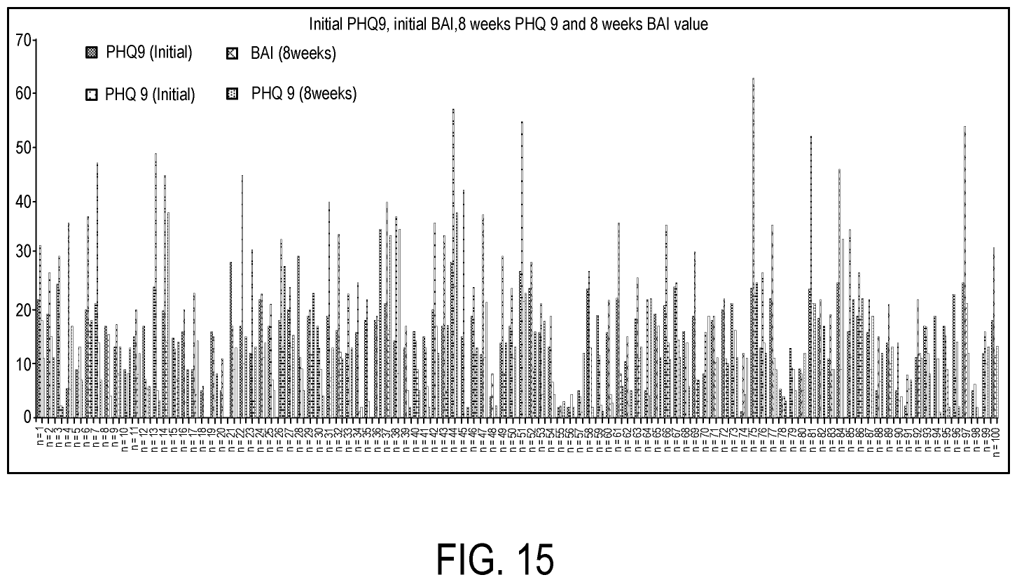

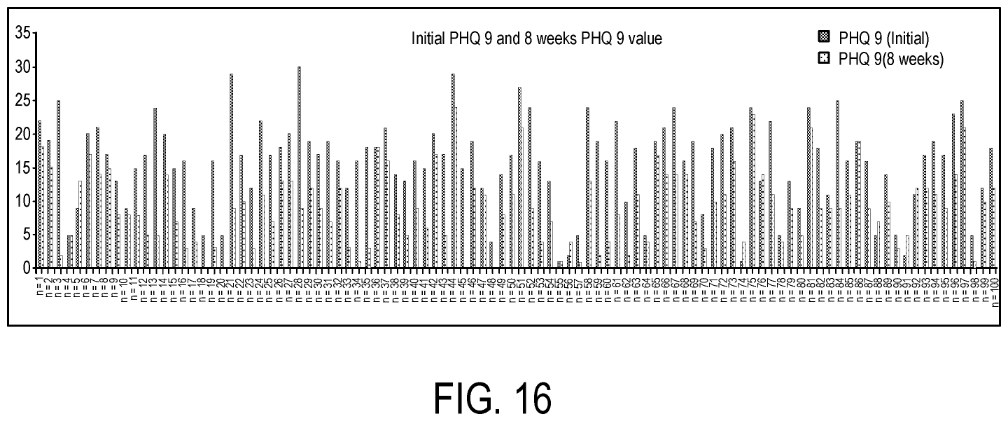

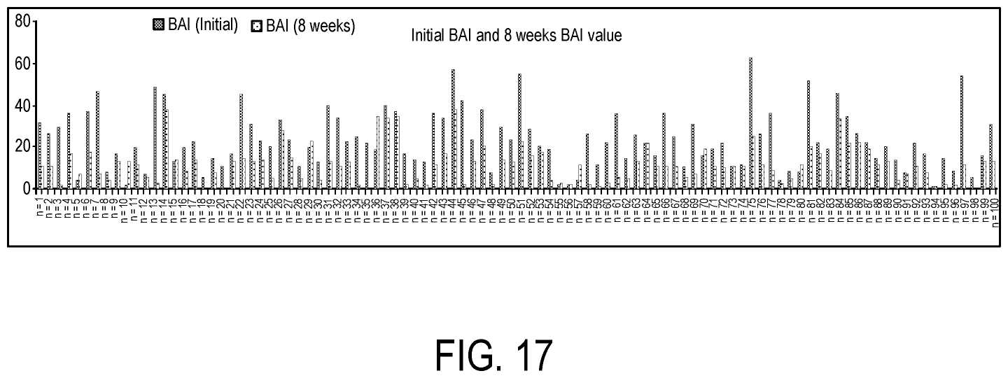

The correlation of initial PHQ-9, initial BAI, 8 weeks PHQ-9 and 8 weeks BAI is shown in the table shown in FIG. 13. The results show a correlation coefficient between PHQ9 Initial and BAI Initial of 0.567 (Sig. 0.000), PHQ9 Initial and PHQ9 8 weeks of 0.620 (Sig. 0.000), PHQ9 Initial and BAI 8 weeks of 0.265 (Sig. 0.008), BAI Initial and PHQ9 8 weeks of 0.501 (Sig. 0.000), BAI Initial and BAI 8 weeks of 0.548 (Sig. 0.000), and finally PHQ-9 8 weeks and BAI 8 weeks of500 (sig. 0.000). Here all the significance values of correlation coefficient were less than 0.01. So, there is strong positive correlation between all the variables. Pair sample t-Tests were calculated to determine the difference between the variables, and the result are shown in the table of FIG. 14. The t-stat value of PHQ9 Initial and BAI Initial was 6.770 (Sig. 0.000), PHQ9 8 weeks and BAI 8 weeks was 3.288 (Sig. 0.001), PHQ9 Initial and PHQ9 8 weeks (Sig. 0.000) and BAI Initial and BAI 8 weeks was 10.241 (Sig. 0.000). In all cases the significance value was less than 0.01, showing a clear statistically significant difference between each pair of variables.

Conclusion

The experiment result shows that there is a positive correlation between all the variables, when data are increased for normal distribution and a statistically significant difference for the measured variables. So, it may be concluded that ADVYNDRA is safe and effective in improving quality of life outcomes.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the claimed invention.

That which is claimed:

1. A metabolic supplement formulation for the treatment depressive disorders comprising a combination of a vitamin B6 compound, a vitamin B12 compound, a folate, betaine equivalent, N-acetyl-L-tyrosine, and a pharmaceutically acceptable carrier.

2. The metabolic supplement formulation of claim 1, wherein the combination comprises the vitamin B6 compound in an amount of between 1 mg and 100 mg, the vitamin B12 compound in an amount between 100 μg and 2,000 μg, Calcium 5-methyltetrahydrofolate in an amount between 100 μg and 2,000 μg, an anhydrous betaine equivalent in an amount between 10 mg and 500 mg, and the N-acetyl-L-tyrosine in an amount between 10 mg and 500 mg.

3. The metabolic supplement formulation of claim 1, wherein the combination comprises the vitamin B6 compound in an amount of 68 mg, the vitamin B12 compound in an amount of 1,000 μg, the Calcium 5-methyltetrahydrofolate in an amount of 1,000 μg, the betaine equivalent in an amount of 200 mg, and the N-acetyl-L-tyrosine in an amount of 275 mg.

4. The metabolic supplement formulation of claim 1, wherein the vitamin B6 compound is at least one of pyridoxal 5′-phosphate, pyridoxine, pyridoxal, pyridoxomaxine, pyridoxine 5′-phosphate, pyridoxal 5′-phosphate, or pyridoxamine 5′-phosphate.

5. The metabolic supplement formulation of claim 1, wherein the vitamin B6 compound is pyridoxine.

6. The metabolic supplement formulation of claim 1, wherein, the B12 compound is at least one of cyanocobalamin (CN-Cbl), aqua cobalamin, adenosylcobalamin, methyl cobalamin, hydroxycobalamin (HC), cyanocobalamin carbanalide, and 5-o-methylbenzylcobalmin [(5-OmeB-za)CN-Cbl] as well as the desdimethyl, monoethylamide and the methylamide analogues of all of the above. Also included are the various analogues and homologues of cobamamide such as coenzyme B12 and 5-deoxydenosylcobalamin. Other analogues include chlorocobalamin, sulfitocobalamin, nitrocobalamin, thiocyanatocobalamin, benzimidazole derivatives such as 5,6-dichlorobenzimidazole, 5-hydroxybenzimidazole, trimethylbenzimidazole, as well as adenosylcyanocobalamin [(Ade)CN-Cbl], cobalamin lactone, cobalamin lactam and the anilide, ethylamide, monocarboxylic and dicarboxylic acid derivatives of vitamin B12 or analogues of dicarboxylic acid derivatives of vitamin B12.

7. The metabolic supplement formulation of claim 1, wherein the vitamin B12 compound is methyl cobalamin.

8. The metabolic supplement formulation of claim 8, wherein the pharmaceutically acceptable carrier comprises one or more pharmaceutically acceptable excipients.

9. The metabolic supplement formulation of claim 1, wherein the folate is a methyl folate.

10. The metabolic supplement formulation of claim 9, wherein the methyl folate is Calcium 5-methyltetrahydrofolate.

11. The metabolic supplement formulation of claim 1, wherein the one or more pharmaceutically acceptable excipients are at least one of microcrystalline cellulose and/or magnesium stearate.

12. A metabolic supplement formulation for the treatment depressive disorders comprising a combination of:
68 mg vitamin B6 compound;
1 mg vitamin B12 compound;
1 mg Calcium 5-methyltetrahydrofolate;
200 mg anhydrous betaine;
275 mg N-acetyl-L-tyrosine; and
a pharmaceutically acceptable carrier.

13. A formulation for treating a depressive disorder consisting essentially of: 68 mg vitamin B6 compound, 1000 μg vitamin B12 compound, 1000 μg Calcium 5-methyltetrahydrofolate, 200 mg betaine, 275 mg N-acetyl-L-tyrosine and a pharmaceutically acceptable carrier.

14. A formulation consisting essentially of: 68 mg vitamin B6 compound, 1000 μg vitamin B12 compound, 1000 μg Calcium 5-methyltetrahydrofolate, 200 mg betaine, and 275 mg N-acetyl-L-tyrosine and a pharmaceutically acceptable carrier.

16. The formulation of claim 1, wherein anhydrous betaine is the betaine equivalent.

17. The formulation of claim 1, wherein the betaine equivalent is at least one of dimethylglycine, a betaine inner salt in anhydrous and hydrated form, hydrochloric acid, citrate, malate aspartate, sarcosine, a combination of betaine and dimethylglycine, or a combination thereof is used.

18. A method for treating a depressive disorder, comprising selecting a subject with a depressive disorder and administering a therapeutically effective amount of the metabolic supplement formulation of claim 1 to the subject.

19. The method of claim 18, wherein the method comprises administering 2 tablets comprises 545 mg of active ingredients, once per day.

20. The method of claim 18, wherein the formulation consists essentially of:
68 mg vitamin B6 compound;
1 mg vitamin B12 compound;
1 mg Calcium 5-methyltetrahydrofolate;
220 mg anhydrous betaine;
275 mg N-acetyl-L-tyrosine; and
a pharmaceutically acceptable carrier.

21. The method of claim 18 further comprising the steps of selecting a subject diagnosed with a psychiatric disorder consisting of major depression, or an anxiety disorder prior to the administration, thereby treating the subject for the depression or the anxiety disorder.

22. The method of claim 18, wherein the folate is Calcium 5-methyltetrahydrofolate.

23. The method of claim 18, wherein the vitamin B6 compound is at least one of pyridoxal 5′-phosphate, pyridoxine, pyridoxal, pyridoxomaxine, pyridoxine 5′-phosphate, pyridoxal 5′-phosphate, or pyridoxamine 5′-phosphate.

24. The method of claim 18, wherein, the B12 compound is at least one of cyanocobalamin (CN-Cbl), aqua cobalamin, adenosylcobalamin, methyl cobalamin, hydroxycobalamin (HC), cyanocobalamin carbanalide, and 5-o-methylbenzylcobalmin [(5-OmeB-za)CN-Cbl] as well as the desdimethyl, monoethylamide and the methylamide analogues of all of the above. Also included are the various analogues and homologues of cobamamide such as coenzyme B12 and 5-deoxydenosylcobalamin. Other analogues include chlorocobalamin, sulfitocobalamin, nitrocobalamin, thiocyanatocobalamin, benzimidazole derivatives such as 5,6-dichlorobenzimidazole, 5-hydroxybenzimidazole, trimethylbenzimidazole, as well as adenosylcyanocobalamin [(Ade)CN-Cbl], cobalamin lactone, cobalamin lactam and the anilide, ethylamide, monocarboxylic and dicarboxylic acid derivatives of vitamin B12 or analogues of dicarboxylic acid derivatives of vitamin B12.

25. The method of claim 18, wherein the vitamin B12 compound is methyl cobalamin.
